(19) United States
(12) Patent Application Publication
(10) Pub. No.: US 2018/0116610 A1
(43) Pub. Date: May 3, 2018

(54) MEDICAL DEVICE CUSTOMIZATION

(71) Applicant: Smiths Medical ASD, Inc., Plymouth, MN (US)

(72) Inventors: Grant ADAMS, Anoka, MN (US); Larry ZALESKY, Shoreview, MN (US); Michael WELSCH, Stillwater, MN (US)

(73) Assignee: Smiths Medical ASD, Inc., Plymouth, MN (US)

(21) Appl. No.: 15/561,912

(22) PCT Filed: Mar. 14, 2016
(86) PCT No.: PCT/US2016/022322

§ 371 Date: Sep. 26, 2017

Related U.S. Application Data
(60) Provisional application No. 62/139,289, filed on Mar. 27, 2015.

(51) Int. Cl. A61B 5/00; A61M 5/14; A61M 5/142

(52) U.S. Cl. CPC A61B 5/7475; A61M 5/1413; G06F 3/0202; A61M 2205/505; A61M 2205/3569; A61M 5/142

(57) ABSTRACT

A medical device includes a placeable control panel and a medical device control module configured to provide at least one of a therapeutic or a patient monitoring function. The placeable control panel can include a surface that includes button markings and a responder wirelessly interrogateable to report at least application information of the placeable control panel. The medical device control module can include an outer surface configured to receive the placeable control panel such that the button markings of placeable control panel are accessible to a user. The medical device control module also can include an interrogator configured to wirelessly interrogate the responder of the placeable control panel for the application information of the placeable control panel and a controller operatively coupled to the interrogator. The controller can be programmed and configured to control the medical device control module in accordance with the application information of the placeable control panel.

### CROSS-REFERENCE TO RELATED APPLICATIONS

The present application is a National Phase entry of PCT Application No. PCT/US2016/022322, filed on Mar. 14, 2016, which claims priority to US Provisional Patent Application No. 62/139,289, filed on Mar. 27, 2015, which are hereby fully incorporated herein by reference.

### TECHNICAL FIELD

This disclosure relates to medical devices, and more particularly, to systems and methods for customization of medical devices.

### BACKGROUND

Medical device manufacturers often wish to supply numerous configurations of a medical device based upon a single hardware platform. The multiple configurations could, for example, be differentiated by the specific applications to which they are directed. For example, a manufacturer could produce an infusion pump platform that could be used for epidural infusions, peripheral intravenous infusions, enteral feeding therapy, as well as any other suitable applications. For each application the manufacturer might wish to provide a pump customized for the specific application. While the underlying physical hardware could be the same for the various pumps, customized configurations could vary in terms of application software, user interface, and physical appearance (for example, color codes or conventions are sometimes used to denote certain medical applications). Devices could also be differentiated by the market for which they are intended, which could require, for example, information to be displayed in a particular language, or for the device to be controlled in compliance with local laws or regulations.

For medical device manufacturers, providing multiple configurations of a single type of product can present financial, logistical and quality challenges. It would therefore be desirable to provide improved systems and methods for customizing medical devices for particular applications and markets.

### SUMMARY

This disclosure relates to medical devices, and more particularly, to systems and methods for customization of medical devices.

In an illustrative but non-limiting example, the disclosure provides a medical device that includes a first placeable control panel and a medical device control module configured to provide at least one of a therapeutic or a patient monitoring function. The first placeable control panel can include a major surface that includes one or more button markings and a responder wirelessly interrogateable to report at least application information of the first placeable control panel. The medical device control module can include an outer surface configured to receive the first placeable control panel such that the one or more button markings of first placeable control panel are accessible to a user. The medical device control module also can include an interrogator configured to wirelessly interrogate the responder of the first placeable control panel for at least the application information of the first placeable control panel and a controller operatively coupled to the interrogator. The controller can be programmed and configured to control the medical device control module in accordance with the application information of the first placeable control panel.

In some cases, the outer surface of the medical device control module can include a control area that underlies at least one of the one or more button markings of first placeable control panel. The control area can be configured to detect at least one of touches or presses at the at least one of the one or more button markings of the first placeable control panel, and can be operatively coupled to the controller and configured to communicate detected touches or presses to the controller.

In some instances, the first placeable control panel can further include one or more touch- or press-sensitive switches corresponding to at least some of the one or more button markings of the major surface. The responder of the first placeable control panel can be configured to respond to wireless interrogation with a status of the one or more touch- or press-sensitive switches. The interrogator of the medical device control module can be configured to wirelessly interrogate the responder of the first placeable control panel for the status of the one or more touch- or press-sensitive switches. The controller of the medical device control module can be programmed and configured to control the medical device control module in accordance with the status of the one or more touch- or press-sensitive switches.

In some cases, responder of the first placeable control panel is a Near Field Communication (NFC) tag. In other cases, the responder of the first placeable control panel is a Radio Frequency IDentification (RFID) tag.

The medical device, in some cases, can further include a second placeable control panel. The second placeable control panel can include features much like the first placeable control panel, but the responder of the second placeable control panel can be wirelessly interrogateable to report at least application information of the second placeable control panel that differs from the application information of the first placeable control panel. The outer surface of the medical device control module can be configured to receive the second placeable control panel (as well as the first), and the controller of the medical device control module can be programmed and configured to control the medical device control module in accordance with the application information of the second placeable control panel (as well as the first). In some cases, the one or more button markings of the first placeable control panel can differ from the one or more button markings of the second placeable control panel.

In some instances, the medical device can further include a placeable localization label including a responder wirelessly interrogateable to report localization information of the placeable localization label. The medical device control module can be configured to receive the placeable localization label. The interrogator of the medical device control module, or another interrogator of the medical device control module, can be configured to wirelessly interrogate the responder of the placeable localization label for the localization information, and the controller of the medical device control module can be programmed and configured to control the medical device control module in accordance with the localization information of the placeable localization label.

In some cases, the first and/or second placeable control panel can include a non-volatile re-writable memory in which at least some of the application information is able to be stored.

In another illustrative but non-limiting example, the disclosure provides a modular control panel that includes a major surface that includes one or more button markings and a responder wirelessly interrogateable to report at least application information of the modular control panel. The modular control panel can be configured to attach to a medical device control module that is configured to provide at least one of a therapeutic or a patient monitoring function. The medical device can include an outer surface configured to receive the modular control panel on an exterior portion of the medical device control module such that the one or more button markings of modular control panel are accessible to a user. The medical device also can include an interrogator and a controller operatively coupled to the interrogator. The interrogator can be configured to wirelessly interrogate the responder of the modular control panel for at least the application information of the modular control panel, and the controller can be programmed and configured to control the medical device control module in accordance with the application information of the modular control panel.

In some instances, the modular control panel can include one or more touch- or press-sensitive switches corresponding to at least some of the one or more button markings of the major surface. The responder of the modular control panel can be configured to respond to wireless interrogation with a status of the one or more touch- or press-sensitive switches, and the interrogator of the medical device control module can be configured to wirelessly interrogate the responder of the modular control panel for the status of the one or more touch- or press-sensitive switches. The controller of the medical device control module can be programmed and configured to control the medical device in accordance with the status of the one or more touch- or press-sensitive switches.

In some cases, the responder can be a passive device configured to receive electrical energy wirelessly from the interrogator.

In some instances, the modular control panel can include a non-volatile re-writable memory in which at least some of the application information is able to be stored.

In still another illustrative but non-limiting example, the disclosure provides a method for producing a customized medical device. The method can include manufacturing a first control panel, manufacturing a second control panel, and manufacturing a generic medical device. The first control panel can include a major surface that includes one or more button markings and a responder wirelessly interrogateable to report at least application information of the first control panel. The second control panel can include a major surface that includes one or more button markings and a responder wirelessly interrogateable to report at least application information of the second control panel, where the application information of the second control panel differs from the application information of the first control panel. The generic medical device can include an outer surface configured to receive either of the first or the second control panels on an exterior portion of the generic medical device such that the one or more button markings of the received control panel are accessible to a user. The generic medical device also can include an interrogator and a controller operatively coupled to the interrogator. The interrogator can be configured to wirelessly interrogate the responder of the received control panel for at least the application information of the received control panel. The controller can be programmed and configured to operate the medical device in a first application configuration when the interrogator receives application information from the first control panel, and to operate the medical device in a second application configuration when the interrogator receives application information from the second control panel. The method can include mating the generic medical device with one of the first control panel and the second control panel, thereby customizing the generic medical device to operate in the application configuration corresponding to the mated control panel.

In some instances, the first control panel can include a non-volatile re-writable memory and the generic medical device can be a drug-delivery device. The method can further include, after manufacture of the first control panel, a user programming the non-volatile re-writable memory of the first control panel with particular application information related to a particular drug delivery. Mating the generic medical device can include mating the generic medical device with the first control panel, thereby customizing the generic medical device to operate in the application configuration corresponding to the first control panel, in accordance with the particular application information programmed by the user.

In some cases, the method for producing a customized medical device can further include manufacturing a localization label and mating the generic medical device with the localization label. The localization label can include a responder wirelessly interrogateable to report localization information of the localization label. The interrogator of the generic medical device can be configured to wirelessly interrogate the responder of the localization label for the localization information of the localization label. The generic medical device can include an information display, and the controller of the generic medical device can be programmed and configured to display information on the information display in a language corresponding to the localization information, such that the mating of the generic medical device with the localization label results in customization of the generic medical device according to the localization information of the localization label.

In some instances, the method can further include de-mating the generic medical device from the one of the first control panel and the second control panel and mating the generic medical device with the other of the first control panel and the second control panel, thereby customizing the generic medical device to operate in the application configuration corresponding to the other of the first control panel and the second control panel.

In some cases, the first and second control panels each can include one or more touch- or press-sensitive switches corresponding to at least some of the one or more button markings of the respective major surfaces. The responder of each control panel can be configured to respond to wireless interrogation with a status of the one or more touch- or press-sensitive switches. the controller of the generic medical device can be programmed and configured to control the medical device in accordance with the status of the one or more touch- or press-sensitive switches of the mated control panel.

In yet another illustrative but non-limiting example, the disclosure provides a medical device that includes a first placeable control panel and a medical device control module configured to provide at least one of a therapeutic or a patient monitoring function. The first placeable control panel can include a major surface that includes one or more button markings and a responder wirelessly interrogateable to report at least application information of the first placeable control panel. The medical device control module can include a receptacle configured to receive the first placeable control panel on an exterior portion of the medical device control module such that the one or more button markings of first placeable control panel are accessible to a user. The medical device control module also can include an interrogator and a controller operatively coupled to the interrogator. The interrogator can be configured to wirelessly interrogate the responder of the first placeable control panel for at least the application information of the first placeable control panel, and the controller can be programmed and configured to control the medical device control module in accordance with the application information of the first placeable control panel.

The above summary is not intended to describe each and every example or every implementation of the disclosure. The Description that follows more particularly exemplifies various illustrative embodiments.

### BRIEF DESCRIPTION OF THE FIGURES

The following description should be read with reference to the drawings. The drawings, which are not necessarily to scale, depict examples and are not intended to limit the scope of the disclosure. The disclosure may be more completely understood in consideration of the following description with respect to various examples in connection with the accompanying drawings, in which:

FIG. 1 is a schematic perspective view of an example medical device that can include a control module and an optional cassette;

FIG. 2 is a schematic front view of a generic control module with two alternative control panels that can be mated with the generic control module to result in a customized medical device control module;

FIG. 3 is a flow diagram of a method for manufacturing various configurations of medical devices;

FIG. 4 is a schematic front view of a generic example medical device control module with two alternative control panels that can be mated with the generic control module to result in a customized medical device;

FIG. 5 is a flow diagram of another method for manufacturing various configurations of medical devices; and

FIG. 6 is a schematic view of a back side of an example medical device control module, an application-specific control panel, and a placeable localization label.

### DESCRIPTION

The following description should be read with reference to the drawings, in which like elements in different drawings may be numbered in like fashion. The drawings, which are not necessarily to scale, depict selected examples and are not intended to limit the scope of the disclosure. Although examples of construction, dimensions, and materials may be illustrated for the various elements, those skilled in the art will recognize that many of the examples provided have suitable alternatives that may be utilized.

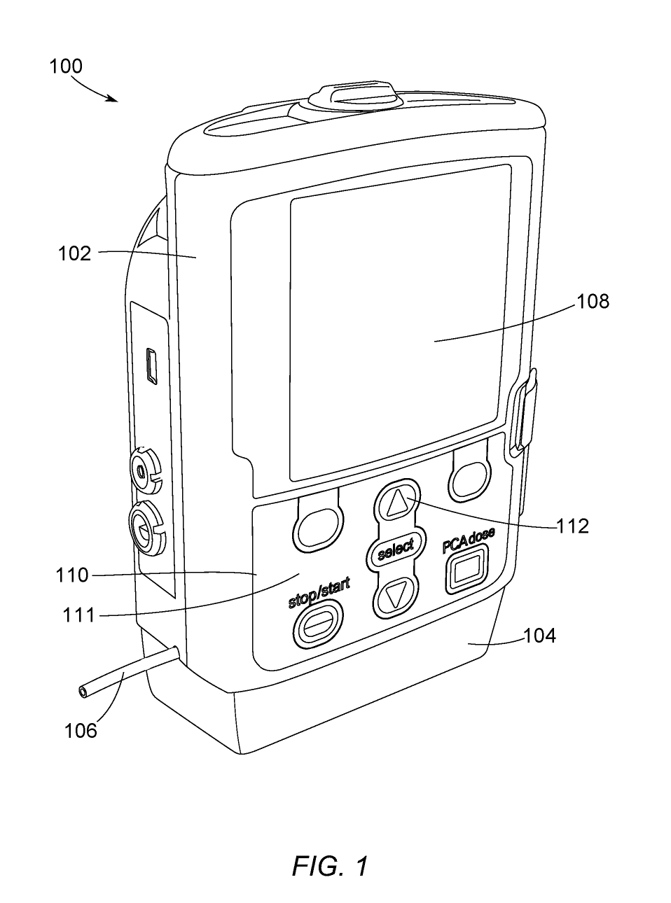

FIG. 1 is a schematic perspective view of an example of a known medical device 100. Medical device 100 may be a CADD® (Computerized Ambulatory Drug Delivery) infusion pump from Smiths Medical ASD, Inc., although the teachings of the present disclosure are not limited to CADD® devices in particular, or infusion pumps in general, and may be practiced with any suitable medical device, such as (but not limited to) warmers, ventilators, or patient monitors. Medical device 100 is an example of a device that can be offered by a manufacturer in numerous configurations but based upon a single base hardware platform. Particulars of various configurations of medical device 100 can be determined by the specific applications and/or markets for which the configurations are intended.

Medical device 100 includes a control module 102 and an optional cassette 104. Optional cassette 104 can be a replaceable reservoir cassette housing a reservoir containing a medicament to be delivered to a patient. Tubing 106 can extend from the cassette 104 and communicate with an infusion set or catheter (not shown) to deliver the medication to the patient. The control module 102 can be used to control the flow of medication from the cassette. Control module 102 in conjunction with a replaceable cassette 104 that is a reservoir cassette can comprise components of a medicament delivery system.

Control module 102 of medical device 100 can include a user interface having a display screen 108 and a control panel 110. Control panel 110 can include a major surface 111 having one or more button markings 112, or visual indicia that indicate control areas that can be touched or pressed by a user to provide input to the control module 102. In various configurations and embodiments of medical device 100, the quantity, layout, and function of buttons markings 112 can differ.

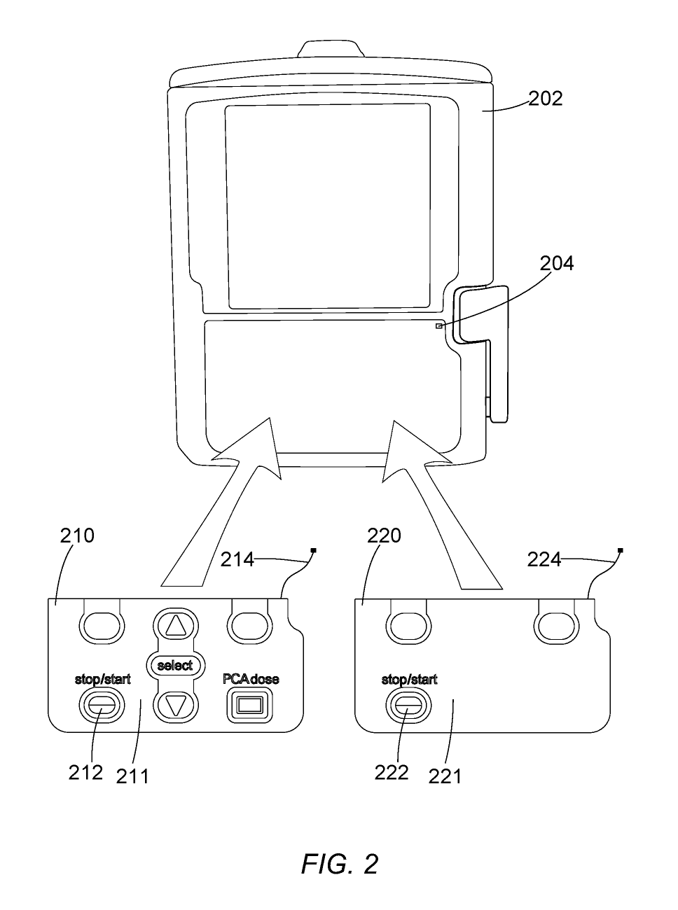

FIG. 2 is a schematic front view of an example of a generic control module 202 with examples of two alternative control panels 210 and 220 (shown apart from generic control module 202). Control panels 210 and 220 can be alternatively mated with generic control module 202 at an outer surface thereof, and configured to receive either control panel 210 or 220 to result in a customized medical device control module similar in visual appearance to known control module 102 of FIG. 1. Control panel 210 can include a major surface 211 having button markings 212 that can differ in arrangement, form, and appearance from button markings 222 on major surface 221 of control panel 220. Each control panel 210, 220 can include switches (for example, membrane switches incorporated into a switch layer of the control panel; not shown) that underlie button markings 212, 222 such that button markings and associated switches together form operable buttons that can receive user input. Each control panel 210, 220 can include a signal cable 214, 224 connected to the switches of the switch layer and connectable to a control panel socket 204 of the generic control module 202 such that the buttons of the control panels can be operatively coupled to a controller (not shown) of the control module via the signal cable and socket.

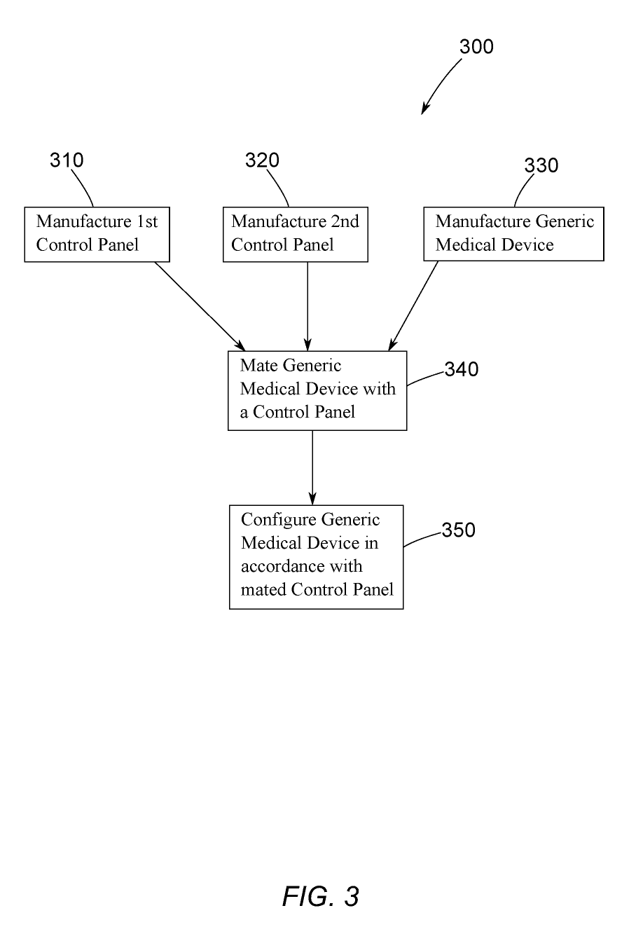

FIG. 3 is a flow diagram of a method 300 for manufacturing various configurations of medical devices. While the description of method 300 refers to the reference numerals of FIG. 2, it should readily be appreciated that the manufacturing concept described is not limited to the control module(s) of FIG. 2 and can be applied to other medical devices. At 310, the method can include manufacturing a first control panel 210. At 320, the method can include manufacturing a second control panel 220. At 330, the method can include manufacturing a generic medical device (e.g., control module) 202. At 340, the method can include mating the generic medical device 202 with a selected one of first control panel 210 and second control panel 220. Mating at 340 can include attaching the signal cable 214 or 224 of the selected control panel 210 or 220 to the control panel socket 204 of the generic medical device 202, and can include attaching (for example, with an adhesive) the selected control panel to the outer surface of the generic medical device. In some cases, attaching the selected control panel 210 or 220 to the outer surface of the generic medical device can include attaching a switch layer of the control panel to the generic medical device, and attaching a layer with the button markings to the switch layer, in any suitable order.

After 340, the physical manufacture of a customized medical device might be completed, but other actions may be needed to produce a finished functional customized medical device. At 350, method 300 can include configuring the generic medical device 202 to operate in accordance with the mated control panel 210 or 220. This can include installing application software into a memory (not shown) of the generic medical device 202 and/or setting values of control parameters of the device. In some cases, these configuration actions of 350 can be manually performed.

After 340 and 350 of method 300, the customized medical device can include a mated control panel 210 or 220 that is tailored for a specific application, and can include application software and/or parameters configured for that specific application and that correspond to the mated control panel. As control panels 210 and 220 can have distinct physical appearances associated with their applications, a properly configured customized medical device can be described as having a visible exterior appearance that matches its (not necessarily visible) internal programming/configuration for its specific application.

It has been recognized, in development of the subject matter hereof, that a hazard might exist in method 300. In particular, steps 340 and 350 may not be performed in correspondence with each other, such that a customized medical device is configured improperly through such erroneous performance of method 300. In such a case, the improperly configured medical device could erroneously have a mated control panel 210 or 220 that imparts an exterior appearance to the medical device that does not match the internal programming/configuration. Obviously, it would be desirable to avoid such misconfigurations. As will be described in more detail below, systems and methods for customizing medical devices, as described by example or otherwise contemplated herein, may also advantageously reduce the possibility of external/internal misconfigurations of customized medical devices.

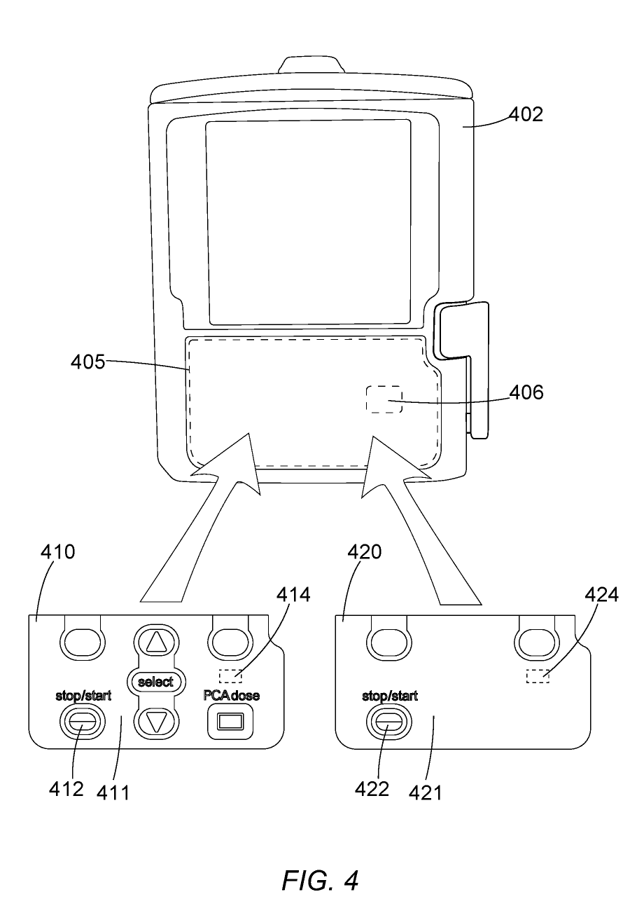

FIG. 4 is a schematic front view of an example of a generic medical device control module 402 with examples of two alternative control panels 410 and 420 (shown apart from the generic medical device control module) that can be alternatively mated with the generic medical device control module at an outer surface configured to receive either of the control panels, to result in a customized medical device. Either of control panels 410 and 420 can be attached to the generic medical device control module 402 with, for example, an adhesive. In some embodiments, the generic medical device control module 402 can include a receptacle configured to receive a control panel 410, 420 on an exterior portion of the control module such that features such as button markings of control panel 410, 420 are accessible to a user. The receptacle can, in some cases, include retention features for the received control panel such as, for example, hooks, tabs, channels, lips, and ledges, which can releaseably secure the control panel to the receptacle in, for example, mating and/or complimentary surface friction fit fashion in an embodiment.

Control panel 410 can include a major surface 411 having button markings 412 that differ from button markings 422 on major surface 421 of control panel 420. Control panels 410, 420 can include touch- or press-sensitive switches (such as membrane switches) underlying the button markings 412, 422, but this is not required in all embodiments. As illustrated in FIG. 4, example control panel 410 can be configured for a patient-controlled analgesia (PCA) application, whereas example control panel 420 could be a simplified control panel directed toward an application with, intentionally, few end-user selectable options.

Control panels 410, 420 can be equipped and configured to communicate wirelessly with medical device control module 402. As such, control panels 410, 420 can be configured without wired signal cables (in contrast to control panels 210, 220), although this is not limiting and in some configurations control panels 410, 420 could include signal cables. Each of control panels 410 and 420 can include a wireless responder 414, 424 (represented in dashed outlines) that is interrogateable (i.e., configured to respond to interrogation) by an interrogator 406 (represented in dashed outlines) of the medical device control module 402. The interrogator 406 and responders 414, 424 can use any suitable communication technology and/or protocol(s). For example, they can use Bluetooth, Wi-Fi, ZigBee, Near Field Communication (NFC), Radio Frequency IDentification (RFID), IrDA, other imaging or non-imaging optical technology, or any other suitable technology. In some embodiments, optical technology is not employed for interrogator-responder communication. In some embodiments, radio frequency technology is not employed for interrogator-responder communication. In some embodiments, non-time varying electric and/or magnetic fields are not employed for interrogator-responder communication.

In some embodiments, the responders 414, 424 can be so-called “passive” devices (sometimes referred-to as “tags”) configured to receive electrical energy wirelessly from the interrogator 406 (“reader”), such that they do not necessitate having a separate power supply, such as an energy storage unit like a battery, nor a wired power connection to (for example) the medical device 402. RFID and NFC technologies can be employed for passive responders 414, 424, but this is not limiting. Interrogator 406 of medical device control module 402 and responders 414, 424 of control panels 410, 420 can be positioned such that the interrogator can reliably read the responder of a control panel when the panel is mated to the control module. In some embodiments, the physical properties of the interrogator 406 and responders 410, 420 can be specified, selected, or otherwise implemented such that the interrogator may not necessarily reliably read a responder of a control panel that is not mated to the medical device control module 402.

Responders 414, 424 can be configured to respond to interrogation with any suitable information. Responders 414, 424 can report application information for their control panels 410, 420. For example, control panels 410, 420 could be mateable to a medical device control module 402 that is an infusion pump, with one of the panels being directed toward epidural infusion (having button markings appropriate for epidural applications and having, for example, a yellow color scheme that is commonly identified by medical practitioners as pertaining to epidural procedures), and the other panel being directed toward enteral feeding (having button markings appropriate for enteral applications and having an orange color scheme that is commonly identified by medical practitioners as pertaining to enteral procedures). It should be noted that these color associations are not universal and may vary between hospitals, institutions, regions, practices, etc. The button layouts shown in FIG. 4 are merely examples and are not necessarily intended to represent appropriate button layouts for these applications. When mated to medical device control module 402, the responder 414, 424 of mated control panel 410, 420 can report application information to interrogator 406 specifying that it is, for example, an epidural or enteral control panel. A controller (not shown) of the medical device control module 402 can be operatively coupled to the interrogator 406, such that it can receive the application information from the interrogator. The controller can be programmed and configured to control the medical device control module 402 in accordance with the application information of the control panel 410, 420. A memory (not shown) operatively coupled to the controller can store application software for any or all applications corresponding to control panels 410, 420, and potentially for any other control panels configured to mate to and operate with medical device control module 402. Medical device control module 402 can include a communication interface (not shown) operatively coupled to the controller. The communication interface can be any suitable hardware/software that enables the controller to communicatively connect to any appropriate external information resource(s) by any appropriate communication technology and/or protocol, etc. The communication interface can enable the controller to connect to an external information system (not shown) such as an electronic medical record (“EMR”) system, an electronic medical administration record (“EMAR”) system, a Hospital Information System (“HIS”), a general purpose personal computer, or another appropriate computing system.

In embodiments where control panels 410, 420 include switches underlying the button markings 412, 422, the responder 414, 424 can be configured to respond to wireless interrogation with the status of the switches. In general, the responder 414, 424 can be configured to respond to wireless interrogation with response bits (i.e., digital bits) of information. In some embodiments, designated bits of the responder's 414, 424 response bits (for example, the first four bits) can encode the application information for the control panel 410, 420. In embodiments where switches underlie the button markings 412, 422, bits of the response bits other than those used to encode the application information can be used to report the status of the switches. In some other embodiments, configurations can be contemplated where response bits used to report the status of the switches can also be used to encode application information.

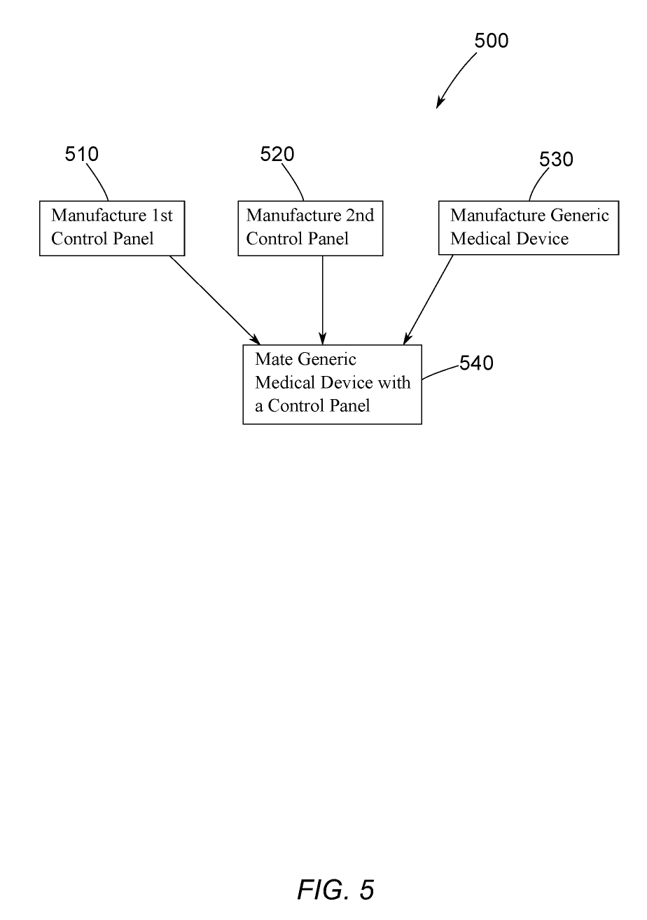

FIG. 5 is a flow diagram of a method 500 for manufacturing various configurations of medical devices. While the description of method 500 refers to the reference numerals of FIG. 4, it should readily be appreciated that the manufacturing concept described is not limited to the medical device(s) of FIG. 4 and can readily be generalized to other medical devices. At 510, the method can include manufacturing a first control panel 410, with the first control panel including a major surface 411 that includes one or more button markings 412 and a responder 414 wirelessly interrogateable to report at least application information of the first control panel. At 520, the method can include manufacturing a second control panel 420, with the second control panel including a major surface 421 that includes one or more button markings 422 and a responder 424 wirelessly interrogateable to report at least application information of the second control panel that differs from application information of the first control panel. At 530, the method can include manufacturing a generic medical device control module 402.

Manufacturing generic medical device control module 402 at 530 can include programming the controller of the control module (for example, by loading or otherwise placing application software into memory for execution by the controller) such that the controller is configured to be able to operate the medical device in either of a first or second application configuration when the interrogator receives application information from either the first or second control panel 410, 420, respectively. The generic medical device control module 402 further can be programmed and configured to operate the medical device in additional other configurations beyond the first and second configurations when the interrogator receives application information from control panels other than the first or second control panels 410, 420 corresponding to the additional other configurations. In some embodiments, manufacturing generic medical device control module 402 can include programming the controller and/or memory of the control module with application software or the like for all possible application configurations corresponding to all control panels that can be mated with, or are anticipated might be mated with, the control module.

At 540, method 500 can include mating the generic medical device control module 402 with one of the first control panel 410 and the second control panel 420. Mating at 540 can include attaching (for example, with an adhesive) the selected control panel to the outer surface of the generic medical device 402. In comparison with action 340 of method 300, for control panels 410, 420 that do not include a signal cable, mating at 540 may include not attaching a signal cable to the generic medical device control module 402, simplifying the attachment. In some embodiments, mating at 540 can be complete with attachment of the selected control panel to the outer surface of the generic medical device 402.

As compared with method 300, in method 500, after mating at 540 has been performed, the medical device can be considered to be configured in accordance with the mated control panel, whereas in method 300, it may be necessary to take further deliberate action(s) at 350 to complete configuration of the medical device in accordance with the mated control panel. Specifically it is to be understood that in method 500, configuration of the medical device 402 in accordance with the mated control panel 410 or 420 can be performed automatically by the controller of the device based upon application information wirelessly read from the responder 414 or 424 of the mated control panel by the interrogator 406 as aforedescribed.

The controller of medical device 402 can be programmed and configured to handle application information read from a mated control panel 410 or 420 in any suitable manner. In some cases, every interrogation of a responder 414, 424 can include reading of application information, but this is not necessarily contemplated in all embodiments. In some cases, every time application information is read by the interrogator 406, the controller can process the application information and proceed accordingly, but this too it not necessarily contemplated in all embodiments. In some non-limiting examples:

A. Upon a first occurrence of powering-up a medical device control module, application information can be read from a responder of a mated control panel and corresponding application software can be made available and/or designated for use by the controller to operate the medical device.

B. Upon a subsequent occurrence of powering-up a medical device control module, application information can be read from a responder of a mated control panel and corresponding application software can be made available and/or designated for use by the controller to operate the medical device.

C. When application information is read from a responder that conflicts with previous application information according to which the medical device is presently configured, an event such as an alarm, interrupt, alert, verification, etc., can be initiated.

D. If new application information is read from a responder that conflicts with previous application information according to which the medical device is presently configured, application software corresponding to the new application information can be made available and/or designated for use by the controller to operate the medical device if the medical device has been placed in an “accept new application” mode. In some cases, placing the medical device in such an “accept new application” mode may require an active input from a user. In other cases, the medical device can enter an “accept new application” mode automatically under predetermined conditions.

These are just some examples. To the extent that they are not incompatible, examples A., B., C., and D. can be combined with others of A., B., C., and D.

Interrogation/reading of responders 414, 424 by interrogator 406 can be performed with any suitable frequency, such as at least 10, 20, 30, 40, 50, 60, 100 or any other number of times per second. When responders 414, 424 report the status of switches underlying button markings 412, 422, a relatively high frequency of interrogation/reading can contribute to a perception of control responsiveness for a user of the medical device.

In some embodiments, a control panel 410, 420 does not have switches underlying some or all of button markings 412, 422. Sensing/detecting touches or presses of some or all of the button marking(s) 412, 422 can be performed by other means. In some embodiments, and as illustrated in FIG. 4, the outer surface of the medical device control module 402 can include a control area 405 that underlies at least one button marking 412, 422 of a mated control panel 410, 420. The control area 405 can be configured to detect touches or presses of or at the button marking(s) 412, 422 of the mated control panel 410, 420. The control area 405 can be operatively coupled to the controller of the medical device control module 402 and configured to communicate detected touches or presses to the controller. The control area 405 can employ any suitable technology, such as resistive or capacitive touchpad sensing, membrane switches, etc. In some embodiments, detection of touches or presses of or at button marking(s) 412, 422 is performed solely by the control area 405 of the control module 402, and not by switches of mated control panel 410, 420. In such embodiments, responsiveness to actuation of buttons of the control panel 410, 420 does not depend upon interrogation/reading of responders 414, 424, and frequency of interrogation/reading can be less than with devices that depend on interrogation/reading for control responsiveness. In some such embodiments, the control module 402 can be configured such that the interrogator 406 interrogates/reads the responder 414, 424 of a mated control panel 410, 420 only as needed to read the application information of the responder, such as once per power-on cycle.

The present disclosure contemplates further opportunities for medical device customization. Medical device manufacturers may wish to produce versions of devices that are directed toward the same application, but different markets, such as countries or regions having different languages. For example, a manufacturer may produce epidural infusion pumps for the United States and Germany that will function essentially identically, except that they will provide user interfaces in English and German. The two pumps may have identical front control panels, but may be required to display permanent or quasi-permanent labeling in the local language.

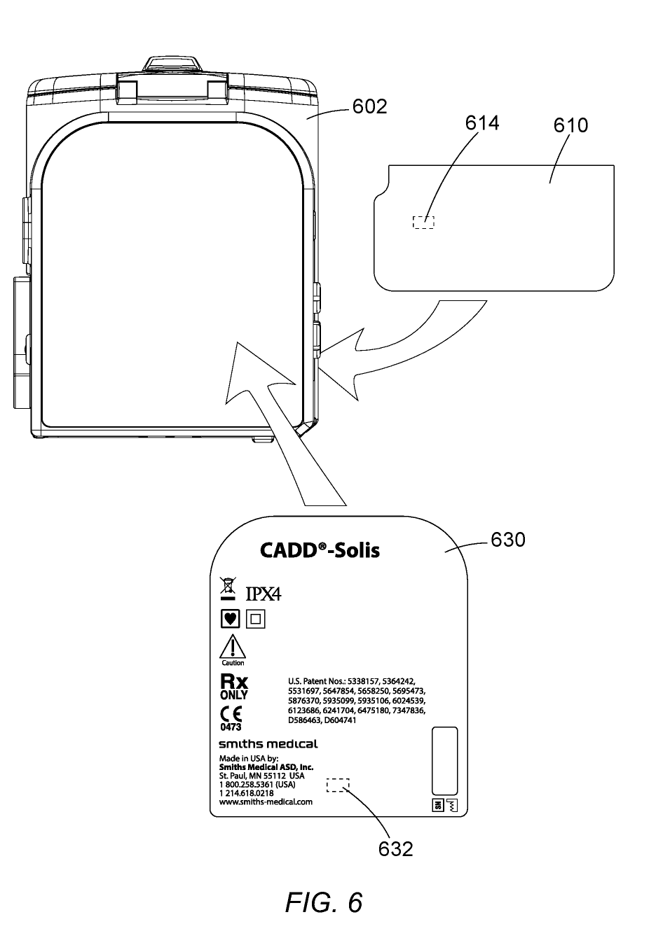

FIG. 6 is a schematic view of a back side of an example medical device control module 602, an application-specific control panel 610 having a responder 614, and a placeable localization label 630. The medical device control module 602 can be, although not necessarily, similar to or the same as medical device control module 402 of FIG. 4, and can be configured to receive the application-specific control panel 610 similar to or the same as control panels 410, 420. The placeable localization label 630 can include a responder 632 wirelessly interrogateable to report localization information of the placeable localization label. The medical device control module 602 can be configured to receive the placeable localization label 630. An interrogator (not shown) of the medical device control module 602 can be configured to wirelessly interrogate the responder 632 of the placeable localization label 630 for the localization information. The interrogator can be the same interrogator that interrogates the responder 614 of control panel 610, or it can be another interrogator. The controller of the medical device control module 602 can be programmed and configured to control the medical device in accordance with the localization information of the placeable localization label 630, for example, by providing user-interface elements on a display in the local language corresponding to the localization information, and/or by operating the medical device in compliance with local laws or regulations, etc.

In some embodiments, responder 614 of control panel 610 can report localization information when interrogated, either in the absence of, or in addition to, localization information of a localization label. The controller of the medical device control module 602 can be programmed and configured to control the medical device in accordance with the localization information of the control panel 610. In cases where both the control panel 610 and localization label 630 provide localization information via their respective responders 614, 632, the controller of the medical device can be programmed and configured to check for agreement in such localization information, and control the medical device in a predetermined manner if a discrepancy is recognized—for example, by indicating an error or fault.

Application-specific control panel 610 is indicated as being receivable on the front side of medical device control module 602, and placeable localization label 630 is indicated as being receivable on the back side, but these locations are not limiting and any suitable locations on control modules can be used for any embodiments of the present disclosure.

Systems and methods of the present disclosure can provide manufacturers ways to customize medical devices that can simplify assembly processes and reduce or remove possible sources for error before the medical devices are delivered to customers and users. The present disclosure further contemplates systems and methods that can provide users with customization options. While control panels such as panels 410, 420, and 610 can be mated to medical device control modules 402, 602 by the manufacturer, in some embodiments they can be attached by users, for example at a healthcare facility such as a hospital. In some embodiments, a first control panel can be mated to a medical device control module and the customized device deployed for a first application. Subsequently, the first control panel can be replaceably removed or de-mated from the medical device control module and a second control panel mated to the control module, followed by deployment in a second application. This could provide a healthcare organization flexibility in their management of their fleet of medical devices. Such control panel mating and de-mating could also be performed by a manufacturer to repurpose medical devices after their initial manufacture.

A variety of workflow scenarios are contemplated in the present disclosure. A control panel such as panels 410, 420, and 610 can include any suitable memory in which to store application information. Application information can be written to memory by the device manufacturer, identifying the control panel as being directed toward, for example, an epidural application or an enteral application. Subsequent to customization of a medical device by way of Method 500 of FIG. 5, an end user such as a nurse can program the medical device via the device's user interface (entering information such as parameters for a specific therapy for a particular patient) and then use the device to provide therapy, such as delivering a medicament.

In addition, the present disclosure contemplates further opportunities to simplify workflows via device customization. In some embodiments, control panel such as panels 410, 420, and 610 can include a non-volatile re-writable memory (not shown) in which at least some of the application information can be stored. Application information can be written, recorded, or otherwise stored in the non-volatile re-writable memory in any suitable manner, by any suitable person or authorized user. It is contemplated that application information written to memory of a control panel by a manufacturer can, in some embodiments, be supplemented with particular application information, which can be written to non-volatile re-writable memory by a user at a time after manufacture.

In one example, a pharmacist at a hospital, in response to a prescription for a drug infusion for a patient, starts with a control panel that is pre-programmed by the manufacturer with application information identifying the control panel as being for use in peripheral infusions. The pharmacist can program the non-volatile re-writable memory of the control panel with particular application information related to the specific drug infusion of the prescription. The particular application information could be any suitable information, such as medication safety limits, particular infusion parameters (such as the name of the drug, infusion volume per time, duration of the infusion, and the like), hospital room number, information related to patient information, etc. The pharmacist could send the custom programmed control panel along with the drug to the hospital room where the drug is to be administered to the patient. The control panel can be attached to a generic infusion pump at the hospital room (or another location) to customize the pump for the particular drug infusion. In some cases, the drug reservoir/container can also include a responder or other communication device readable by the pump, such that the pump can verify that the control panel and drug reservoir correspond before initiating infusion.

In another embodiment, a control panel directed toward a particular application can be attached to a disposable or replaceable component of a medical device. For example, a control panel having button markings, switches underlying the button markings, and a wireless responder (i.e., a configuration similar to control panels 410, 420) could be attached to a cassette similar to cassette 104 of medical device 100, with the cassette having a reservoir of a medicament to be delivered in the particular application of the control panel. The control panel attached to the cassette could be programmed by a pharmacist with particular application information related to the medicament delivery. The physical attachment of a control panel that is directed toward, and is particularly programmed for, the medicament cartridge to which it is attached could reduce the possibility of misconfigurations and hazards resulting therefrom.

In some embodiments, a medical device control module can receive application information from a control panel via interrogation that results in the medical device downloading additional information from an external source, such as a hospital information system. The additional information can be any appropriate information pertinent to the operation of the medical device, such as application software, medication safety information and/or limits, parameters for a particular use or therapy such as a drug delivery to be performed by the device, and so on. In other embodiments, a medical device, having received application information from a control panel via interrogation, can be configured and ready to perform its intended medical function without necessarily receiving any further information from an external source.

The present disclosure contemplates the possibility of medical devices operating with any suitable additional devices having wireless responders, other than the control panels such as control panels 410, 420, 610 and localization labels such as localization label 630 aforedescribed. The additional devices could be removably or non-removably attached to a control module, and could be another label or an additional or auxiliary control panel. For example, the user interface of a medical device having a simplified control panel such as control panel 420 could be augmented by an optional auxiliary control panel that could be attached to the medical device in addition to the simplified primary control panel. In some cases, an additional information device having a responder could be configured not to attach the control module, but could be transiently brought into proximity to the interrogator of the control module to enable information to be transferred from its responder.

The addition devices other than control panels and localization labels could provide, via their responders, any suitable application information, or non-application information to a medical device. Furthermore, control panels and localization labels could provide non-application information via their responders. As just one example of non-application information, a responder of a control panel, localization label, or additional device could provide network configuration information to a medical device that allows it to configure its communication interface to communicate via a network with which it previously was not configured to communicate. In another example, a responder of a control panel, localization label, or additional device could trigger a medical device to download additional information from an external source, such as a hospital information system or any other suitable information system, which could be application information or non-application information.

In some embodiments, a control panel has button markings, switches underlying the button markings, and a signal cable to attach to a medical device control module when the control panel is mated to the control module. The control panel can have application information that can be read by the control module over the signal cable. These embodiments combine the physical connection (via the signal cable) of control panels like panels 210, 220 with the application information described in relation to panels 410, 420, 610.

In some embodiments, a control panel and/or localization label having a responder readable by an interrogator of a medical device control module to provide application and/or localization information can be part of a (re)placeable housing or portion of housing of a medical device, such that the medical device can be customized for an application by attaching the housing or housing portion to the medical device, possibly after removing a previously attached housing or housing portion.

This disclosure is to be understood to be not limited to the particular examples described herein, but rather should be understood to cover all aspects of the disclosure and equivalents thereof. Various modifications, processes, and components, as well as numerous structures to which the disclosure can be applicable, will be readily apparent to those of skill in the art upon review of the instant specification.

1. A medical device, comprising:
a first placeable control panel, including:
a major surface that includes one or more button markings; and
a responder wirelessly interrogateable to report at least application information of the first placeable control panel; and
a medical device control module configured to provide at least one of a therapeutic or a patient monitoring function, including:
an outer surface configured to receive the first placeable control panel such that the one or more button markings of first placeable control panel are accessible to a user;
an interrogator configured to wirelessly interrogate the responder of the first placeable control panel for at least the application information of the first placeable control panel; and
a controller operatively coupled to the interrogator, the controller programmed and configured to control the medical device control module in accordance with the application information of the first placeable control panel.

2. The device of claim 1, wherein the outer surface of the medical device control module includes a control area that underlies at least one of the one or more button markings of first placeable control panel, the control area configured to detect at least one of touches or presses at the at least one of the one or more button markings of the first placeable control panel, the control area further being operatively coupled to the controller and configured to communicate detected touches or presses to the controller.

3. The device of claim 1, wherein:
the first placeable control panel further includes one or more touch- or press-sensitive switches corresponding to at least some of the one or more button markings of the major surface;
the responder of the first placeable control panel is configured to respond to wireless interrogation with a status of the one or more touch- or press-sensitive switches;
the interrogator of the medical device control module is configured to wirelessly interrogate the responder of the first placeable control panel for the status of the one or more touch- or press-sensitive switches; and
the controller of the medical device control module is programmed and configured to control the medical device control module in accordance with the status of the one or more touch- or press-sensitive switches.

4. The device of claim 1, wherein the responder of the first placeable control panel is a Near Field Communication (NFC) tag.

5. The device of claim 1, wherein the responder of the first placeable control panel is a Radio Frequency IDentification (RFID) tag.

6. The device of claim 1, further comprising:
a second placeable control panel, including:
a major surface that includes one or more button markings; and
a responder wirelessly interrogateable to report at least application information of the second placeable control panel, the application information of the second placeable control panel differing from the application information of the first placeable control panel;
wherein the outer surface of the medical device control module is configured to receive the second placeable control panel, and
wherein the controller of the medical device control module is programmed and configured to control the medical device control module in accordance with the application information of the second placeable control panel.

7. The device of claim 6, wherein the one or more button markings of the first placeable control panel differ from the one or more button markings of the second placeable control panel.

8. The device of claim 1, further comprising a placeable localization label, the placeable localization label including a responder wirelessly interrogateable to report localization information of the placeable localization label;
wherein the medical device control module is configured to receive the placeable localization label;
wherein the interrogator of the medical device control module, or another interrogator of the medical device control module, is configured to wirelessly interrogate the responder of the placeable localization label for the localization information; and
the controller of the medical device control module is programmed and configured to control the medical device control module in accordance with the localization information of the placeable localization label.

9. The device of claim 1, wherein the first placeable control panel includes a non-volatile re-writable memory in which at least some of the application information is able to be stored.

10. A modular control panel, comprising:
a major surface that includes one or more button markings; and
a responder wirelessly interrogateable to report at least application information of the modular control panel;
wherein the modular control panel is configured to attach to a medical device control module that is configured to provide at least one of a therapeutic or a patient monitoring function, the medical device including:
an outer surface configured to receive the modular control panel on an exterior portion of the medical device control module such that the one or more button markings of modular control panel are accessible to a user;
an interrogator configured to wirelessly interrogate the responder of the modular control panel for at least the application information of the modular control panel; and
a controller operatively coupled to the interrogator, the controller programmed and configured to control the medical device control module in accordance with the application information of the modular control panel.

11. The modular control panel of claim 10, further comprising one or more touch- or press-sensitive switches corresponding to at least some of the one or more button markings of the major surface,
wherein the responder of the modular control panel is configured to respond to wireless interrogation with a status of the one or more touch- or press-sensitive switches;
further wherein the interrogator of the medical device control module is configured to wirelessly interrogate the responder of the modular control panel for the status of the one or more touch- or press-sensitive switches; and
the controller of the medical device control module is programmed and configured to control the medical device in accordance with the status of the one or more touch- or press-sensitive switches.

12. The modular control panel of claim 10, wherein the responder is a passive device configured to receive electrical energy wirelessly from the interrogator.

13. The modular control panel of claim 10, further comprising a non-volatile re-writable memory in which at least some of the application information is able to be stored.

14. A method for producing a customized medical device, comprising:
manufacturing a first control panel, the first control panel including:
a major surface that includes one or more button markings; and
a responder wirelessly interrogateable to report at least application information of the first control panel;
manufacturing a second control panel, the second control panel including:
a major surface that includes one or more button markings; and
a responder wirelessly interrogateable to report at least application information of the second control panel, the application information of the second control panel differing from the application information of the first control panel;
manufacturing a generic medical device, the generic medical device including:
an outer surface configured to receive either of the first or the second control panels on an exterior portion of the generic medical device such that the one or more button markings of the received control panel are accessible to a user;
an interrogator configured to wirelessly interrogate the responder of the received control panel for at least the application information of the received control panel; and
a controller operatively coupled to the interrogator, the controller programmed and configured:
to operate the medical device in a first application configuration when the interrogator receives application information from the first control panel; and
to operate the medical device in a second application configuration when the interrogator receives application information from the second control panel; and
mating the generic medical device with one of the first control panel and the second control panel, thereby customizing the generic medical device to operate in the application configuration corresponding to the mated control panel.

15. The method of claim 14, wherein:
the first control panel includes a non-volatile re-writable memory; and
the generic medical device is a drug-delivery device;
the method further including:
after manufacture of the first control panel, a user programming the non-volatile re-writable memory of the first control panel with particular application information related to a particular drug delivery,
wherein mating the generic medical device includes mating the generic medical device with the first control panel, thereby customizing the generic medical device to operate in the application configuration corresponding to the first control panel, in accordance with the particular application information programmed by the user.

16. The method of claim 14, further comprising:
manufacturing a localization label, the localization label including a responder wirelessly interrogateable to report localization information of the localization label;
mating the generic medical device with the localization label,
wherein the interrogator of the generic medical device is configured to wirelessly interrogate the responder of the localization label for the localization information of the localization label;
wherein the generic medical device includes an information display; and
wherein the controller of the generic medical device is programmed and configured to display information on the information display in a language corresponding to the localization information, such that the mating of the generic medical device with the localization label results in customization of the generic medical device according to the localization information of the localization label.

17. The method of claim 14, further comprising:
de-mating the generic medical device from the one of the first control panel and the second control panel; and
mating the generic medical device with the other of the first control panel and the second control panel, thereby customizing the generic medical device to operate in the application configuration corresponding to the other of the first control panel and the second control panel.

18. The method of claim 14, wherein:
the first control panel further includes one or more touch- or press-sensitive switches corresponding to at least some of the one or more button markings of the major surface;
the responder of the first control panel is configured to respond to wireless interrogation with a status of the one or more touch- or press-sensitive switches;
the second control panel further includes one or more touch- or press-sensitive switches corresponding to at least some of the one or more button markings of the major surface;
the responder of the second control panel is configured to respond to wireless interrogation with a status of the one or more touch- or press-sensitive switches; and
the controller of the generic medical device is programmed and configured to control the medical device in accordance with the status of the one or more touch- or press-sensitive switches of the mated control panel.

19. A medical device, comprising:
a first placeable control panel, including:
a major surface that includes one or more button markings; and
a responder wirelessly interrogateable to report at least application information of the first placeable control panel; and
a medical device control module configured to provide at least one of a therapeutic or a patient monitoring function, including:
a receptacle configured to receive the first placeable control panel on an exterior portion of the medical device control module such that the one or more button markings of first placeable control panel are accessible to a user;
an interrogator configured to wirelessly interrogate the responder of the first placeable control panel for at least the application information of the first placeable control panel; and
a controller operatively coupled to the interrogator, the controller programmed and configured to control the medical device control module in accordance with the application information of the first placeable control panel.
